(19) United States
(12) Patent Application Publication
(10) Pub. No.: US 2010/0076272 A1
(43) Pub. Date: Mar. 25, 2010

(54) INTRODUCED IN CUTANEOUS TRANSLUMINA APPARATUS

(76) Inventor: Jose Humberto Yatsuda MOROMIZATO, Sao Paulo (BR)

Correspondence Address: GOTTLIEB RACKMAN & REISMAN PC, 270 MADISON AVENUE, 8TH FLOOR, NEW YORK, NY, 10016-0601, US

(21) Appl. No.: 12/493,453

(22) Filed: Jun. 29, 2009

(30) Foreign Application Priority Data
Sep. 25, 2008 (BR) PI 0804147-4

(51) Int. Cl. A61B 1/06

(52) U.S. Cl. 600/249

(57) ABSTRACT

An apparatus (1), of the type used to achieve extra-body view of veins during medical-hospital procedures, more specifically, such apparatus is of the type constituted by a portable body (2), with base (3) and cover (4) duly fitted and affixed (5), conforming the internal space to hold an electronic circuit board(P) fed by electricity (cable) or battery (B), whilst one of its extremities provides at least two light rods (6), each of which is equipped with groups of three or more Leds (7) driven by a on/off switch (8), placed in one of the faces of the body (2); both the base (3) and the cover (4) provide, in one of the co-flat extremities between both, receptacles (3a) and (4a) which constitute cylindrical berths (B), provided with holders (10) for axial laying of each of the terminals (11) of each corresponding light rod (6), which are able to rotate up to 90 degrees, axial and angularly (α), in each of the berths (BE); the improvements introduced in the translumina apparatus (1) were especially developed for use in members of reduced diameter (M) such as low weight newly borns, children up to 7 kilograms, preferably and also small size animals, since the angular and independent adjustment (α) of each lighting head (6) allows the professional to adjust the light flows of the Leds (7) to any diameter of member (M).

### TECHNICAL FIELD

The present invention deals with improvements introduced in a cutaneous transiumina apparatus, of the type used to achieve extra-body view of veins during medical-hospital and lab procedures, more specifically, such translumina has been especially developed for use in neonatology and similar conditions, including small size animals, since the translumina apparatus is equipped with two articulated rods which allows the achievement of certain angles for the light flows, covering diameters of members not reached with the conventional apparatus.

### BACKGROUND

It is known to those qualified in the health area that veins and arteries are widely used to collect blood for exams, and serve as means for the application of fluid medications, of the electrolytic types, nutrition and general drugs. Generally, such procedures are initially done by means of a syringe or needle of a catheter, however, for such procedures to happen safely, it is necessary, above all, that the professional identifies the best vein to act.

There are numerous types of patients which show high degree of difficulty to find the veins, such as obese persons, elder persons, diabetics, those having dark skin, newly borns, persons under chemotherapy, among other.

In order to improve the location of the veins before starting the procedures, the professionals of the health area already have apparatus connected by cutaneos translumina, which, as a rule, comprise a manual size apparatus fed by electricity (cable) or battery, capable of energizing at least one rod equipped with three or more Leds which preferably adopt a color combination between red and yellow, and are assembled with a slight separation. Such rods with the Leds are placed over the patient's skin and are lit by a simple on/off switch, producing intense light flows inside the patients subcutaneous tissue. The veins, containing venous blood which color is dark, absorbe the light and are seen as dark lines, enhanced from the patient's skin tone, whether it is pink, black or brown.

Thus, the member receiving the translumina allows the professional to clearly view the veins of such member, causing the professional to act in a fast and precise manner, eliminating a series of inconveniences which are so well known in the state of the technique.

The currently known translumina apparatus are presented in several versions and models, and may be used in patients under several conditions, such as UTIs, surgical centers, ambulatories and even in children.

However, as known by those acting in the area, particularly in the case of newly born or very thin children the diameter of a member, for instance, a newly born arm which may measure up to 1.5 cm diameter, that is, is extremely small, makes it very difficult to use the action reach of the light flows of the Leds, since, being assembled in rigid rods or only one rod with electrical wire feed, the member does not receive the translumina in a correct manner (in the two opposite sides), making it necessary for the professional to improvise, or, as happens in the majority of occasions, uses its own experience, taking a much longer time as compared to the use of the translumina.

The petitioner already has a patent request in course before the INPI, no. PI 0301185-2, which refers to a portable translumina, with two light rods and which has all commands friendly to who operates the apparatus. Such apparatus has been achieving good results during its use, however has the inconveniences reported in the previous paragraph, that is, has rigid rods, and does not cover the necessities in respect to newly borns and other patient in similar conditions.

The same happens with the translumina apparatus of the type found in the foreign documents, for instance, the North-American document US 20050257795, which shows some rod models, however non of them allows the approximation of the action reach in members with lower diameters, even one of them providing two angular and fixed rods in a semi-opened line, however not allowing for the regulation, which limits its application to newly borns.

Another well known apparatus, sold under the commercial name “Venoscan”, comprises a small head with semicircular form, which has, in each free and flat extremity, a place where the Leds are uniformly assembled. However, being the head made by a rigid plastic part, the action reach of the Leds is ideal for members with larger diameter, for instance, adults. Even if such head was resized to act in children and newly borns, it would be difficult for the professional to operate.

### BRIEF DESCRIPTION OF THE INVENTION

In face of the above, the petitioner, acting in the area of medical-hospital equipment and apparatus, submits the improvements in cutaneous translumina, which were especially developed to act in members of reduced diameter, especially newly borns, children in general and even small size pets or wild animals, which has two articulated rods in their corresponding basis, projecting out of the main body, which articulated rods are independent one from another, allowing easy manual adjustment by the professional, which may, during the procedure, find the bet angle of the rods, so that the light flows act correctly on the member where the procedure is performed.

### DESCRIPTION OF THE DRAWINGS

Supplementing the present description so as to obtain a better understanding of the characteristics of the present invention and according to a preferred practical realization thereof, a set of drawings accompanies the description, where there are examples, without limitation, of the following:

FIG. 1 represents an exploded perspective view of the innovated translumina apparatus, showing all components, it so being that one of its articulated rods is shown exploded and in cut, in amplified detail, while the other is shown fit into the support terminal;

FIG. 2 represents the apparatus in assembled position and covered by an ancillary cover, of the type equipped with metallic bolt for affixation to the belt or pocket;

FIG. 3 shows a lateral view of the apparatus, without the ancillary cover;

FIG. 4 shows an inferior view of the translumina apparatus;

FIG. 5 represents an inferior perspective with the rods in angular move; and

FIGS. 6 and 7 are front views of the apparatus, it so being that FIG. 6 shows the rods aligned in parallel and FIG. 7 show the articulated rods in angle, in relation to a member of reduced diameter.

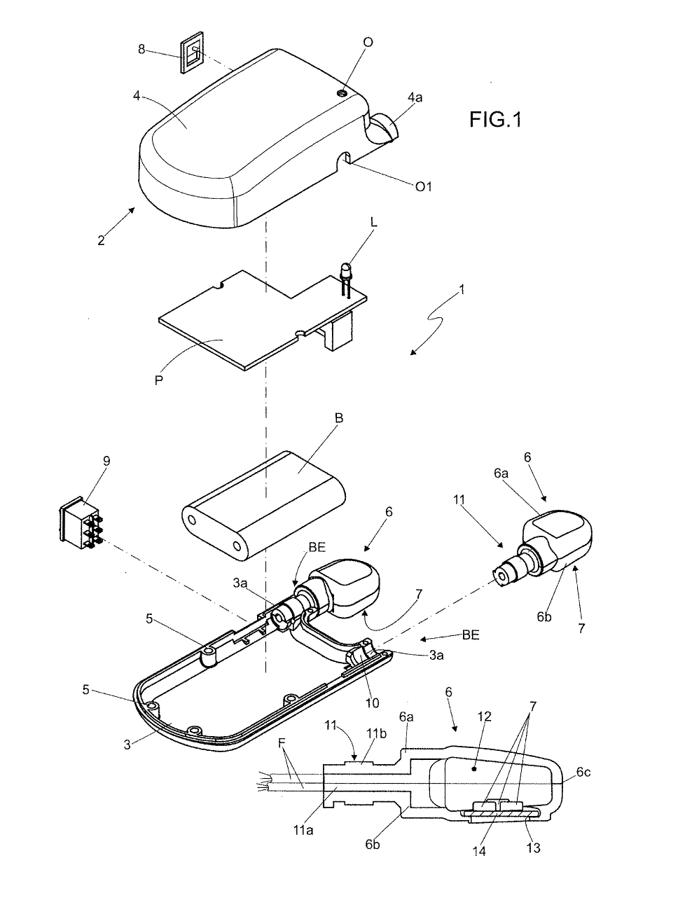

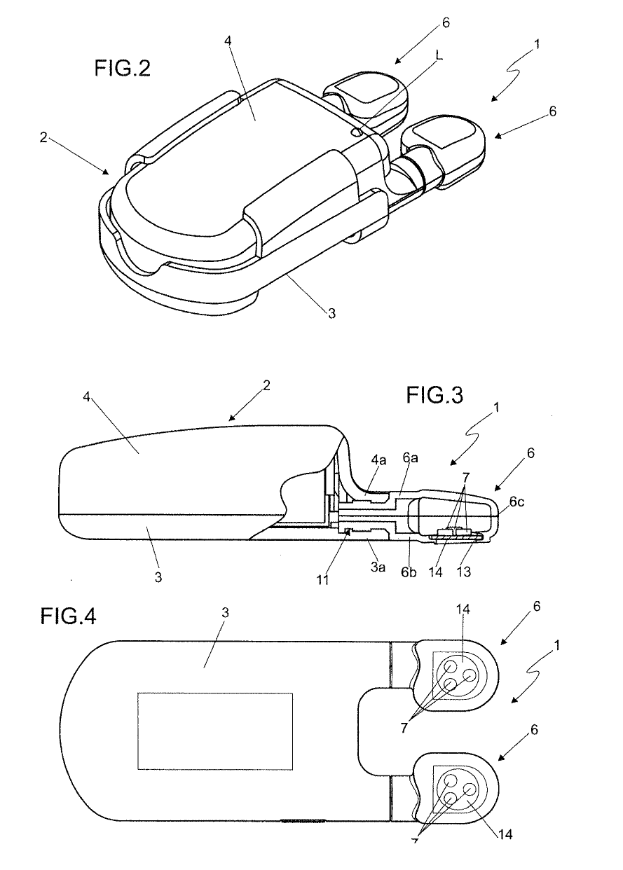

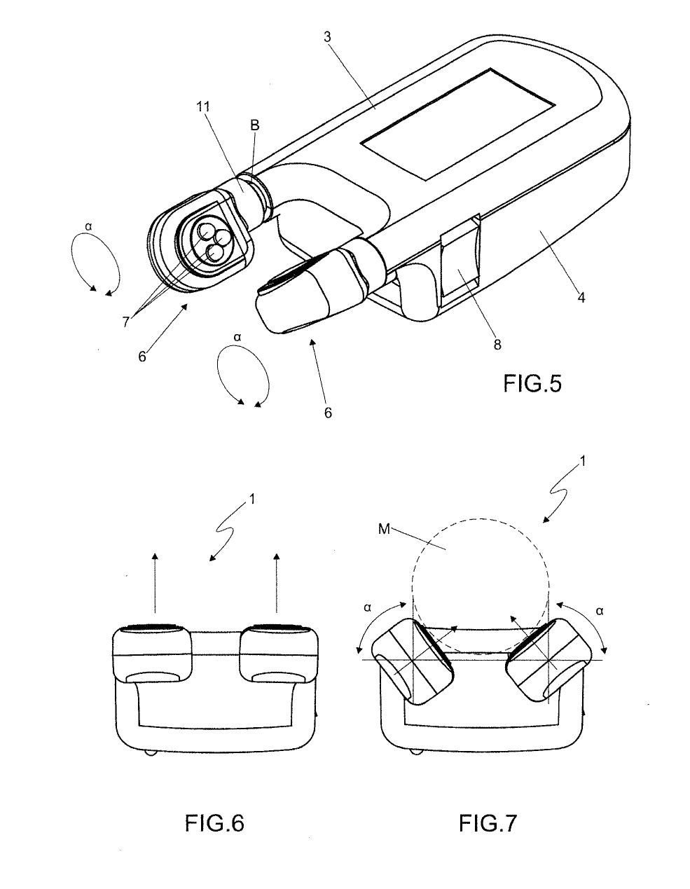

### DETAILED DESCRIPTION OF THE INVENTION

Referring now to the accompanying drawings, the present invention refers to “IMPROVEMENTS INTRODUCED TO CUTANEOUS TRANSLUMINA APPARATUS” (1), of the type used for extra body view of veins during medical, hospital and lab procedures. Particularly, such apparatus is constituted by one portable body (2), that is, base (3) and cover (4), duly fit and affixed (5) one to another, so as to allow the handling by one of the professional's hand, which body (2) bears, internally, an electronic circuit board(P) with operational components (not illustrated) and at least one signaling Led (L), which can be viewed in orifice (O), made to cover (4), being such circuit board(P) fed by electricity (cable) or battery (B), preferably of the reloading type, and where such cable (not illustrated) can access the battery through whole (01), provided on the cover side (4), of one of its body extremities (2), at least two light rods are projected (6), each equipped with a group of three or more Leds (7) where the color combination uses, preferably, red and white and are driven by an on/off switch (8) placed in one of the body faces (2), which is responsible for driving an electric contact (9).

According to the present invention, both the base (3) and the cover (4) provide, in one of the co-flat extremities between them, receptacles (3a) and (4a) which are cylindrical berths (BE) equipped with holders (10) for the axial placement of each of the terminals (11) of each corresponding light rods (6), which can rotate, both axial and angularly (α) in each of the berths (BE).

Each light rod (6) is comprised of two parts, being a top one (6a) and a bottom one (6b), both united and linked to each other through the contact boards (6c), so as to comprise two main sectors, being one sector called fitting terminal (11) and one sector called receptacle (12) of the Leds (7) which, on its side, is equipped with a hole (13) where a concentration and amplifying lens is set (14) for the light flows of the Leds (7).

The terminal fitting area (11) is internally piped (11a) for the crossing of wires (F) which feed the Leds (7), whilst, externally, it is equipped with a central thickening (11b), able of fitting the retention (11) of each berth (BE), allowing the axial and angular rotation (α), without the light rod being able to escape (6) from the corresponding berth (B).

The improvements introduced to the translumina apparatus (1) were especially developed for use in members of reduced diameter (M) such as newly borns, children up to 7 years old, preferably and also small size animals, since the angular and independent adjustment (α) of each lighting rod (6) allows the professional to adjust the light flows of the Leds (7) to any diameter of member (M).

It is certain that, when the present invention is placed in use, there may be introduced changes regarding certain construction and form details, without this implying to departing from the essential principles which are clearly consubstantiated in the claims chart, being it understood that the terminology used had descriptive and not limitation purpose.

1. An apparatus for the exterior viewing of the blood vessels, including the veins and arteries of a person during medical-hospital procedures, said apparatus comprising:
a portable body (2), with a base (3) and cover (4) fitted and affixed thereto to define an internal space, an electronic circuit board(P) disposed in said inner space and receiving electric power, said body having two cylindrical berths housing respective two light rods (6) a, each light rod (6) being equipped with groups of three or more Leds (7) receiving selectively power from the electronic circuit board (P) and arranged and constructed to illuminate an organ disposed adjacent to said rods;
wherein said rods have a longitudinal axis and are rotatable within said berth about said longitudinal axis by an angle α of up to 90 degrees.

2. The apparatus according to claim 1 characterized by the fact that each light rod (6) is comprised of a rod enclosure formed by two parts including a superior part (6a) and an inferior part(6b), said parts being united and linked to each other through a contact edges (6c), said housing forming fitting end (11) disposed in said berth and a section forming a receptacle (12) for receiving the Leds (7), said section further including an amplifying lens placed in the path of the light flow of the Leds (7).

3. The apparatus of claim 2 wherein said fitting end (11) is arranged to carry wires (F) feeding the Leds (7) and externally equipped with a central thickening (11b), said berth being formed with an indent receiving said central thickening (11b) to allow said light rods to rotate around their respective longitudinal axes.

4. The apparatus according to claim 1 wherein said rods rotate independently of each other.
